(19) United States
(12) Patent Application Publication
(10) Pub. No.: US 2024/0363251 A1
(43) Pub. Date: Oct. 31, 2024

(54) GENETIC SCREENING TO DETERMINE HEALTH RISKS ASSOCIATED WITH INTERACTING PHENOTYPES

(71) Applicant: Helix, Inc., San Mateo, CA (US)

(72) Inventor: Elizabeth Cirulli Rogers, Lakeside, CA (US)

(21) Appl. No.: 18/141,009

(22) Filed: Apr. 28, 2023

(51) Int. Cl. G16H 50/30; G16B 20/20; G16B 20/40

(52) U.S. Cl. CPC G16H 50/30; G16B 20/20; G16B 20/40

(57) ABSTRACT

Systems and methods herein provide for identifying phenotypes that impact disease progression for carriers of genetic variants. One method includes identifying, from a larger population of gene sequences, a first plurality of gene sequences of probands having a genetic variant that is susceptible to contracting a primary phenotype. For each of a plurality of interacting phenotypes, the method includes identifying, from the first plurality of gene sequences, a second plurality of gene sequences of probands having the interacting phenotype, determining a difference between an odds ratio indicating a likelihood of probands having the primary phenotype for the second plurality of gene sequences and an odds ratio indicating a likelihood of probands having the primary phenotype for the first plurality of gene sequences, and selecting the interacting phenotype based on the difference. The method also includes identifying a risk of contracting the primary phenotype for each of the selected interacting phenotypes.

### BACKGROUND

Genetic screening can reveal genetic variants in people that are more likely to contribute to certain diseases. For example, variants in the BRCA1 and BRCA2 genes have been linked to a substantially increased risk of contracting breast cancer. People having such a genetic variant would generally like to know the risks of contracting a certain disease so that they can make informed decisions about treatment, including lifestyle changes, dietary changes, surgeries, medication, and the like. For example, a patient having a particularly high risk for breast cancer may consider a preemptive mastectomy.

Present genetic screening can provide a patient with a general idea of what the risks of contracting a certain disease are. But these general levels of risk may not always be acceptable to certain patients. For example, the genetic screening may show that one patient's chances of contracting a certain disease may have increased from one in one million to one in one thousand. If that patient has a higher level of risk tolerance, that patient may not seek out treatment even though the patient's risk has increased significantly. Thus, while genetic testing is intended to improve the predictive power for developing a certain disease for a patient, this information may not be considered useful or be acted upon.

Certain phenotypes, such as age, weight, diet, behavioral traits (e.g., smoking, exercising, etc.), and the like can influence the risk of contracting a certain disease associated with a genetic variant, both positively and negatively. And studies have attempted to categorize risk in a manner that considers patient phenotype in combination with genetics. However, determining a more precise level of risk based on these phenotypes generally requires a formal interaction analysis, which is computationally intensive, time intensive, and is typically limited to the consideration of a few phenotypes at a time. Thus, a comprehensive understanding of risk on an individualized basis remains particularly difficult to quantify in any meaningful way.

### SUMMARY

Systems and methods herein provide for identifying phenotypes that impact disease progression for carriers of a genetic variant. By carefully considering the impact of varying phenotypes within carrier populations, the systems and methods presented herein can generate risk profiles which are tailored to each patient. These risk profiles can be orders of magnitude more precise than the risk profiles determined by genetic testing alone. Thus, these systems and methods advantageously provide impactful information to a patient such that a patient can make more informed healthcare decisions. The system and methods presented herein can also identify phenotypes that have additive and multiplicative effects on disease risk.

In one embodiment, a method includes identifying, from a larger population of gene sequences, a first plurality of gene sequences of probands having a genetic variant that is susceptible to contracting a primary phenotype. Then, for each of a plurality of interacting phenotypes, the method includes identifying, from the first plurality of gene sequences, a second plurality of gene sequences of probands having the interacting phenotype, determining a difference between an odds ratio indicating a likelihood of probands having the primary phenotype for the second plurality of gene sequences and an odds ratio indicating a likelihood of probands having the primary phenotype for the first plurality of gene sequences, and selecting the interacting phenotype based on the difference. The method also includes identifying a risk of contracting the primary phenotype for each of the selected interacting phenotypes.

In another embodiment, a non-transitory computer readable medium embodying programmed instructions which, when executed by a processor, are operable for performing a method that includes identifying, from a larger population of gene sequences, a first plurality of gene sequences of probands having a genetic variant that is susceptible to contracting a primary phenotype. For each of a plurality of interacting phenotypes, the method also includes identifying, from the first plurality of gene sequences, a second plurality of gene sequences of probands having the interacting phenotype, determining a difference between an odds ratio indicating a likelihood of probands having the primary phenotype for the second plurality of gene sequences and an odds ratio indicating a likelihood of probands having the primary phenotype for the first plurality of gene sequences, and selecting the interacting phenotype based on the difference. The method also includes identifying a risk of contracting the primary phenotype for each of the selected interacting phenotypes.

In yet another embodiment, a system includes a database of gene sequences of probands. The system also includes a processor that identifies, from a larger population of the gene sequences, a first plurality of gene sequences of probands having a genetic variant that is susceptible to contracting a primary phenotype. For each of a plurality of interacting phenotypes, the process identifies, from the first plurality of gene sequences, a second plurality of gene sequences of probands having the interacting phenotype, determines a difference between an odds ratio indicating a likelihood of probands having the primary phenotype for the second plurality of gene sequences and an odds ratio indicating a likelihood of probands having the primary phenotype for the first plurality of gene sequences, and selects the interacting phenotype based on the difference. The processor also identifies a risk of contracting the primary phenotype for each of the selected interacting phenotypes.

Additionally, the various embodiments disclosed herein may be implemented in a variety of ways as a matter of design choice. For example, some embodiments herein are implemented in hardware, whereas other embodiments may include processes that are operable to implement and/or operate the hardware. Other exemplary embodiments (e.g., methods and computer readable media relating to the foregoing embodiments) may be described below. The features, functions, and advantages that of been discussed can be achieved independently in various embodiments or may be combined in yet other embodiments, further details of which can be seen with reference to the following description and drawings.

### BRIEF DESCRIPTION OF THE FIGURES

Some embodiments of the present invention are now described, by way of example only, and with reference to the accompanying drawings. The same reference number represents the same element or the same type of element on all drawings.

FIG. 1 is a block diagram of an exemplary processing system that is operable to identify disease risk based on genetic variants and various interacting phenotypes.

FIG. 2 is a flowchart of an exemplary process for identifying disease risk based on a genetic variant and various interacting phenotypes.

FIG. 3 is a flowchart of an exemplary process for determining a risk for contracting a disease for an interacting phenotype.

FIG. 4 is a flowchart of an exemplary process for statistically defining a risk for contracting the disease for an interacting phenotype.

FIG. 5 is a graph of an exemplary probability distribution of probands having an interacting phenotype.

FIG. 6 is a graph exemplarily illustrating improved positive predictive values based on an interacting phenotype.

FIG. 7 is a graph exemplarily illustrating improved positive predictive values based on various interacting phenotypes.

FIG. 8 is a graph 800 that exemplarily illustrates the risk profiles of carriers and noncarriers of TTNtvs contracting cardiomyopathy.

FIG. 9 exemplarily illustrates the effects of atrial fibrillation and various tobacco smoking habits on contracting cardiomyopathy for probands that were positive for being carriers of TTNtvs and for probands that were not positive for being carriers of TTNtvs.

FIG. 10 is a block diagram of an exemplary computing system in which a computer readable medium provides instructions for performing methods herein.

### DETAILED DESCRIPTION OF THE FIGURES

The figures and the following description illustrate specific exemplary embodiments. It will thus be appreciated that those skilled in the art will be able to devise various arrangements that, although not explicitly described or shown herein, embody certain principles and are included within the scope of the embodiments. Furthermore, any examples described herein are intended to aid in understanding the embodiments and are to be construed as being without limitation to such specifically recited examples and conditions. As a result, the embodiments are not limited to any of the examples described below.

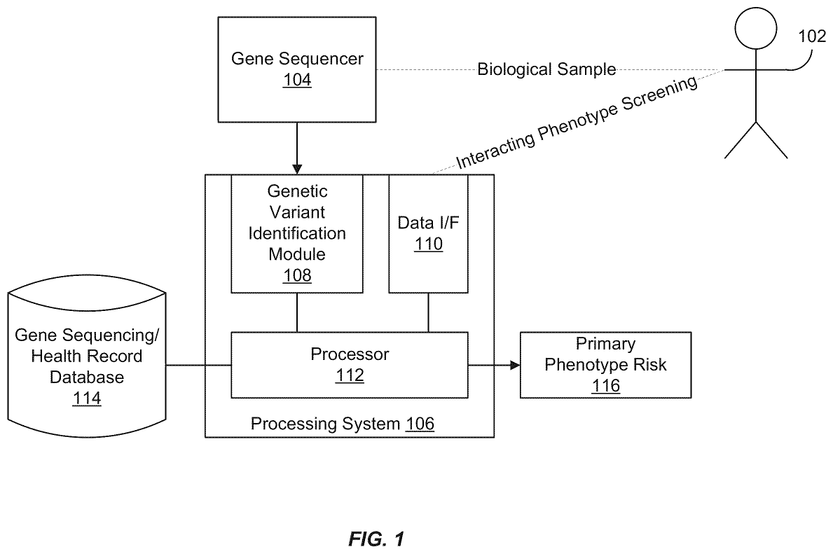

FIG. 1 is a block diagram of a processing system 106 that is operable to identify disease risk based on genetic variants and various phenotypes. In this embodiment, the processing system 106 is operable to retrieve genetic sequences and health records of a plurality of probands (e.g., hundreds of thousands of people of various ages, sexes, and ethnicities) from the database 114. The processing system 106 includes a processor 112 that is operable to identify, from the retrieved genetic sequences and health records, a first group of gene sequences having a genetic variant that is susceptible to contracting a primary phenotype. For example, truncating variants in the TTN gene (a.k.a. TTNtvs) have been linked to the disease of cardiomyopathy, a primary phenotype. The processing system 106 may be instructed to locate probands in the database 114 that have this genetic variant.

Once this group of probands has been identified, the processing system 106 may identify a subset of this group that has a particular interacting phenotype. Interacting phenotypes are generally: quantifiable traits such as age and weight; physiological conditions such as atrial fibrillation, a history of stroke, etc.; and even behavioral patterns such as tobacco smoking (including the age at which smoking began, the frequency of smoking, etc.), diet, exercise patterns, etc. In this part of the process, the processing system 106 may direct the processor 112 to statistically analyze the effects of various interacting phenotypes (e.g., hundreds or even thousands) on the genetic variant contributing to the primary phenotype, sequentially and/or in combination. For example, processing system 106 may analyze a first interacting phenotype to establish its effect on the primary phenotype, and then analyze a second interacting phenotype to establish its effect on the primary phenotype, and so on. Alternatively or additionally, the processing system may analyze a combination of interacting phenotypes, such as smoking and atrial fibrillation, to establish its effect on the primary phenotype.

In any case, the processing system 106 may then determine a difference between an odds ratio indicating a likelihood of probands having the primary phenotype in this group of identified probands and an odds ratio indicating a likelihood of probands having the primary phenotype in the overall population retrieved from the database 114. If this odds ratio difference breaches a predetermined threshold, the interacting phenotype may be selected for further analysis, otherwise it is withdrawn from further analysis. For example, if the odds ratio difference is statistically significant, the interacting phenotype may be used to identify a risk of contracting the primary phenotype.

In this regard, the processing system 106 is operable to identify the specific risk of contracting the primary phenotype for each of a plurality of interacting phenotypes such that a patient can make a more informed decision on lifestyle changes and/or health treatments to reduce the risk of contracting the primary phenotype. For example, once the risks for contracting the primary phenotype have been computed for each interacting phenotype and a specific genetic variant, a future patient 102 is able to submit a biological sample to a gene sequencer 104 to sequence all or part of the patient's genome. The patient 102 may also submit interacting phenotype screening information to the processing system 106 (e.g., via a data interface 110). Then, depending on the genetic screening being performed on the patient 102, the genetic variant identification module 108 may isolate the portion of the patient 102's sequenced genome to determine if the patient 102 has the particular genetic variant of the genetic screening. If the patient 102 does possess the genetic variant, then the processor 112 may compare the patient 102's interacting phenotype screening information to the computed risk profiles to determine which of the risk profiles is similar to the phenotype screening information of the patient 102, and thus determine the risk of the patient 102 contracting the primary phenotype (i.e., element 116).

With this in mind, the processing system 106 is any device, system, software, or combination thereof operable to statistically compute risk profiles for contracting a variety of primary phenotypes linked to various genetic variants. For example, like TTNtvs have been linked to cardiomyopathy, variants in the BRCA1 and BRCA2 genes have been linked to breast cancer. The processing system 106 may be operable to statistically identify which interacting phenotypes may contribute to people contracting breast cancer with the BRCA1 and BRCA2 gene variants, just as the processing system 106 may statistically ID which interacting phenotypes may contribute to cardiomyopathy.

The database 114 is any device, system, software, or combination thereof operable to store or otherwise retrieve gene sequencing data and associated health record information of a plurality of probands. Examples of such include the United Kingdom Bio Bank comprising plink formatted population level exome files for 450,000 individuals and the Healthy Nevada Project comprising 28,423 genetic sequences. Of course, the embodiments herein are not intended to be limited to any particular gene sequencing/health record database.

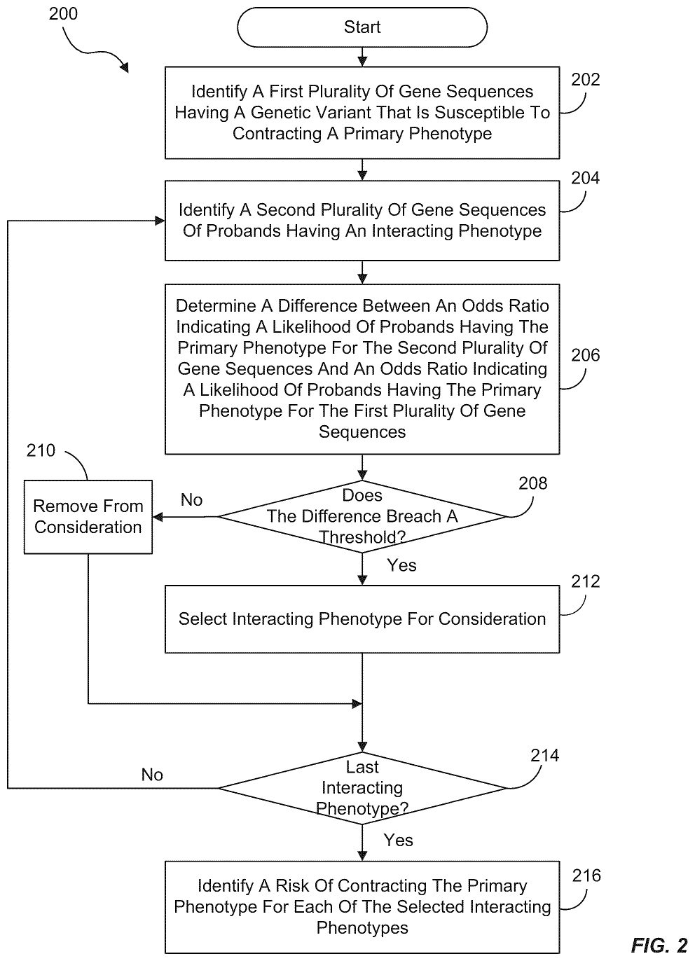

FIG. 2 is a flowchart of an exemplary process 200 for identifying disease risk based on a genetic variant and various interacting phenotypes, as implemented by the processing system 106 of FIG. 1. The embodiments herein are generally computer implemented due to the computational intensity involved in processing hundreds of thousands of genetic sequences. For example, cardiomyopathy is a group of diseases that affect a patient's heart muscle and is considered a primary phenotype (e.g., a medical condition diagnosed by medical personnel). Cardiomyopathy has been linked to a genetic variant in the TTN gene of humans, specifically unique truncating variants in the TTN gene (a.k.a. TTNtvs). The TTN gene is genetically complex with over 364 exons and four major structural regions (i.e., Z-disk, I-band, A-band, and M-band), each with largely independent functional roles. Identifying TTNtvs in the TTN gene is already complex, and this identification is compounded when hundreds of thousands of genetic sequences of probands are compared to one another.

In this embodiment, the processing system 106 retrieves a plurality of genetic sequences of probands (e.g., hundreds of thousands of genetic sequences) to identify a first plurality of gene sequences having a genetic variant that is susceptible to contracting a primary phenotype, in the process element 202. For example, the processing system 106 may access the database 114, and select a group of the probands that have the genetic variant TTNtvs, which again has been linked to cardiomyopathy, a primary phenotype. Then, the genetic variant (or a group of rare genetic variants collapsed together) is evaluated for its predictive power in relation to the primary phenotype according to various techniques, such as regression analysis. In certain scenarios, predictive power is determined by consulting existing medical literature and research.

Then, the processing system 106 may identify a second plurality of gene sequences of the probands having an interacting phenotype, in the process element 204. For example, interacting phenotypes may contribute to a patient contracting a primary phenotype, particularly when the patient has a genetic variant that is known to contribute to the primary phenotype. Again, interacting phenotypes may be quantifiable traits such as age and weight; physiological conditions such as atrial fibrillation, a history of stroke, etc.; and even behavioral patterns such as smoking (including the age at which smoking began, the frequency of smoking, etc.), diet, exercise patterns, etc. Information on interacting phenotypes for the population may be acquired from Electronic Health Records (EHRs) based on diagnosis codes, answers to survey questions, and the like. For example, interacting phenotypes may be indicated within Current Procedural Terminology (CPT), International Classification of Diseases (ICD), or other codes.

The processing system 106 may consider thousands of interacting phenotypes as a part of an analysis process for a primary phenotype and the particular genetic variant. Thus, the process element 204 may be implemented as a sort of “for loop” process (e.g., in serial or parallel) in which a number of interacting phenotypes are selected for analysis in the process 200. And, for each interacting phenotype, the processing system 106 may determine a difference between an experiment odds ratio indicating a likelihood of probands having the primary phenotype in the second plurality of gene sequences and a baseline odds ratio indicating a likelihood of probands having the primary phenotype for the first plurality of gene sequences, in the process element 206. In other words, when analyzing a particular interacting phenotype, the processing system 106 may compare the experiment and baseline odds ratios.

In one embodiment, the baseline odds ratio is the odds ratio of contracting the primary phenotype, as calculated by comparing people with the particular genetic variant (i.e., regardless of interacting phenotype status) to people without the particular genetic variant (i.e., regardless of interacting phenotype status). The baseline odds ratio may be calculated according to the following formula, wherein (a) is the number of people with the particular genetic variant that have the primary phenotype, (b) is the number of people with the particular genetic variant that do not have the primary phenotype, (c) is the number of number of people without the particular genetic variant that have the primary phenotype, and (d) is the number of people without the particular genetic variant that do not have the primary phenotype. Standard error or confidence interval metrics may also be calculated for the odds ratio to facilitate analysis.

$$OR = \frac{( a / b )}{( c / d )} \qquad ( 1 )$$

In one embodiment, the experiment odds ratio is the odds ratio of contracting the primary phenotype, as calculated by comparing people that have the particular genetic variant and also have the interacting phenotype to people without the variant that have the interacting phenotype. The experiment odds ratio may be calculated according to the formula (1), wherein (a) is the number of people with the interacting phenotype and particular genetic variant that have the primary phenotype, (b) is the number of people with the interacting phenotype and particular genetic variant that do not have the primary phenotype, (c) is the number of people without the particular genetic variant that have the interacting phenotype and the primary phenotype, and (d) is the number of people without the particular genetic variant that have the interacting phenotype and that do not have the primary phenotype. Standard error or confidence interval metrics may also be calculated for the odds ratio to facilitate analysis.

The odds ratio of people with the interacting phenotype having a particular disease (i.e., the experiment odds ratio) is compared to the odds ratio of people having the disease in the larger population of carriers of the particular genetic variant in general (i.e., the baseline odds ratio). The processing system 106 may then determine whether this difference breaches a threshold, in the process element 208, to either remove the interacting phenotype from consideration (i.e., the process element 210) or select the interacting phenotype for further consideration with respect to the particular genetic variant (i.e., the process element 212).

This threshold difference between the two odds ratios may regard a statistically significant difference. Statistically significant generally means non-overlapping confidence intervals of the odds ratio (e.g., as determined using regression analysis or other analysis techniques). If the processing system 106 detects a statistically significant difference between the odds ratio for the primary phenotype in the subset and the odds ratio for the primary phenotype in the population of carriers as a whole, then the interacting phenotype may help in evaluating risk for the primary phenotype.

With this in mind, the odds ratio may be considered in order to detect and evaluate phenotypes having an additive or multiplicative impact on risk. Additive and multiplicative effects can also be identified by analyzing the whole dataset together and including the primary phenotype, the interacting phenotype, and the primary phenotype multiplied by the interacting phenotype as covariates. The result of multiplying the primary phenotype by the interacting phenotype is generally referred to as an “interaction phenotype.” However, as the sample size is larger for the whole cohort, this analysis technique may require more compute time than analyzing just a subset.

Alternatively or additionally, even when there is not a statistically significant difference in the odds ratio, if a Positive Predictive Value (PPV) is above a pre-determined threshold, then an interacting phenotype may have an important relationship to risk of contracting the primary phenotype. PPV generally regards the likelihood of an individual with a positive test result truly having a particular gene variant and/or a disease in question, as is the case for atrial fibrillation as an interaction phenotype with variants in the TTN gene. Thus, the processing system 106 may select an interacting phenotype for analysis when the PPV breaches a pre-determined threshold. Conversely, a Negative Predictive Value (NPV) generally regards the likelihood of an individual with a negative test result truly not having a particular gene variant and/or a disease in question. The processing system 106 may select an interacting phenotype for analysis when the NPV breaches another pre-determined threshold.

The processing system 106 may continue this initial analysis of each of the preselected interacting phenotypes until there are no more phenotypes to analyze, in the process element 214. If interacting phenotypes remain, the processing system 106 returns to the process element 204 to analyze the next interacting phenotype. Otherwise, the processing system 106 identifies a risk of contracting the primary phenotype for each of the selected interacting phenotypes, in the process element 216. For example, people generally understand that smoking is an unhealthy choice. But people generally do not understand its specific risk of contracting cardiomyopathy. And people with the genetic variant TTNtvs are much more likely to be at risk for contracting cardiomyopathy. But the specific risk of contracting cardiomyopathy of a tobacco smoker with the inherited genetic variant TTNtvs is largely unknown. The processing system 106 is operable to identify a specific risk of such a person contracting cardiomyopathy. Then, that person could make a more educated decision with regards to smoking tobacco.

It should be noted that when a primary phenotype is associated with both a genotype and an interacting phenotype, their effects can have different mathematical properties, including additive or multiplicative. For example, in an additive relationship, the genotype can have a similar odds ratio with a primary phenotype, regardless of whether it is in the whole population or restricted to those with the interacting phenotype. In a multiplicative relationship, the genotype has an odds ratio with the primary phenotype that is significantly different between the population as a whole and the subset with the interacting phenotype. The processing system 106 is operable to consider both additive and multiplicative risk by comparing the odds ratios and their confidence intervals in both the whole population and in those with the interacting phenotype.

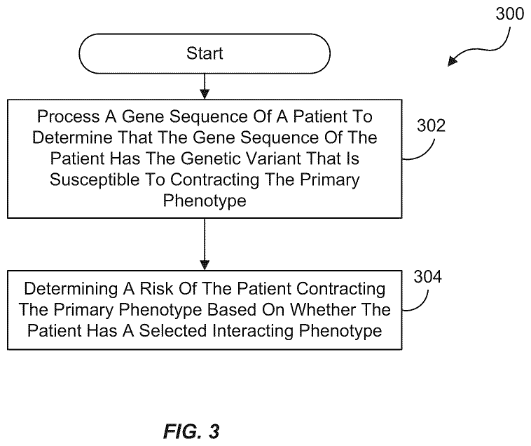

FIG. 3 is a flowchart of an exemplary process 300 for determining a risk for contracting a disease for an interacting phenotype. In this embodiment, the processing system 106 may process a gene sequence of a patient to determine that the gene sequence of the patient has the genetic variant that is susceptible to contracting the primary phenotype, in the process element 302. And, based on the analysis performed in FIG. 2 for the genetic variant, the primary phenotype, and the interacting phenotypes that are comparable with the patient's interacting phenotypes, the processing system 106 may identify a risk of the patient contracting the primary phenotype, in the process element 304.

Some interacting phenotypes are quantitative in nature. That is, some interacting phenotypes may have a range of possible numerical values. These interacting phenotypes may be split into upper and lower ranges, which are separately considered during the analysis. For example, for a quantitative phenotype such as blood pressure, Body Mass Index (BMI), or weight, values within one standard deviation of the mean for the population may be ignored for the purposes of analysis. However, values more than one standard deviation above the mean may be used to create a first subset for analysis. And values more than one standard deviation below the mean may be separately used to create a second subset for analysis. Of course, other cutoffs could be used as a matter of design choice (e.g., 0.5 standard deviation, 1.5 standard deviation, etc.)

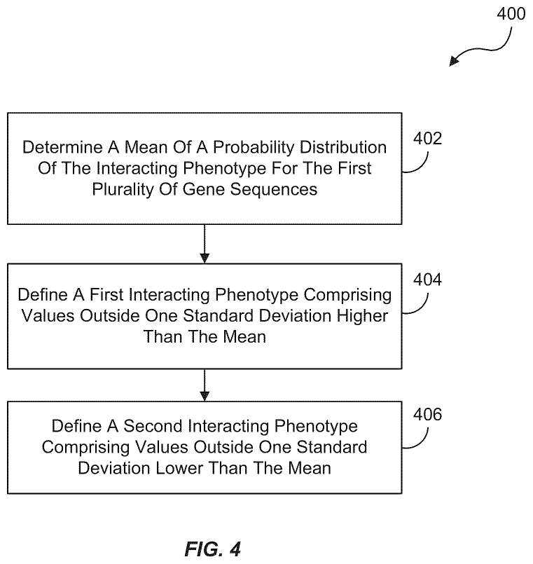

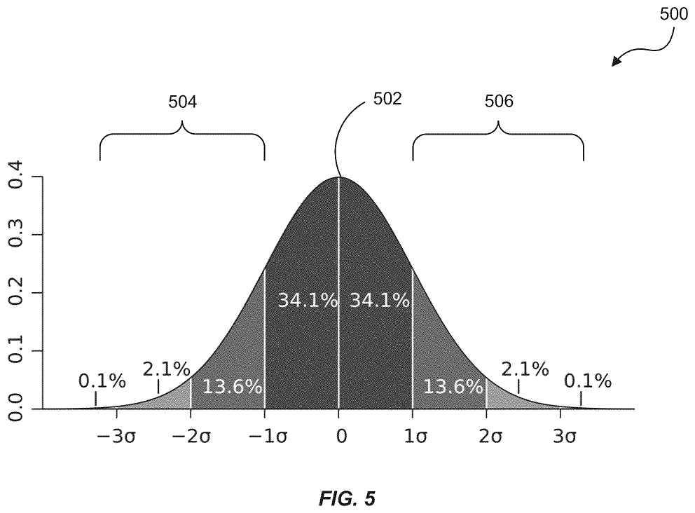

With this in mind, FIG. 4 is a flowchart of an exemplary process 400 for statistically defining a risk for contracting the disease for the phenotype. In this embodiment, the processing system 106 subdivides an interacting phenotype. The processing system 106 does so by first determining the mean of a probability distribution of the interacting phenotype for the first plurality of gene sequences, in the process element 402. Then, the processing system 106 defines a first interacting phenotype comprising values outside one standard deviation higher than the mean, in the process element 404. And then the processing system 106 defines a second interacting phenotype comprising values outside one standard deviation lower than the mean, in the process element 406. Again, different cutoffs can be used to identify the extremes of the distribution for the analysis. An example of such a probability distribution is illustrated in FIG. 5.

FIG. 5 is a graph of an exemplary probability distribution 500 of an interacting phenotype. In this example, the processing system 106 examined 450,000 exomes of the TTN gene to determine carriers of TTNtvs (i.e., probands). The processing system 106 then determined which of the probands had cardiomyopathy (i.e., a primary phenotype), and analyzed those probands with atrial fibrillation (i.e., an interaction phenotype). Then, the processing system 106 analyzed approximately 7,000 interacting phenotypes to observe differences in the main result for cardiomyopathy (e.g., using diagnosis codes, survey questions, etc.). The quantitative phenotypes were determined to be the highest ˜16% and the lowest ˜16% of the population (e.g., one standard deviation and above 506 from the mean 502 and one standard deviation and below 504 from the mean 502, respectively). These values may then be used to determine the PPVs and the NPVs for each interacting phenotype having an effect on the primary phenotype

$$( e . g . , \frac{Number of true positives}{Number of true positives + Number of false positives} and \\ \frac{Number of true negatives}{Number of true negatives + Number of false negatives} ,$$

respectively). Thus, in this example, the PPV and the NPV would each be 84%.

After the processing system 106 completes this analysis, interacting phenotypes having a PPV or a NPV upon the primary phenotype beyond a threshold amount may then be reported out as being influential with respect to risk. This thresholding may select PPVs or NPVs beyond a predefined value, or may select a predefined number of PPVs or NPVs of the highest value.

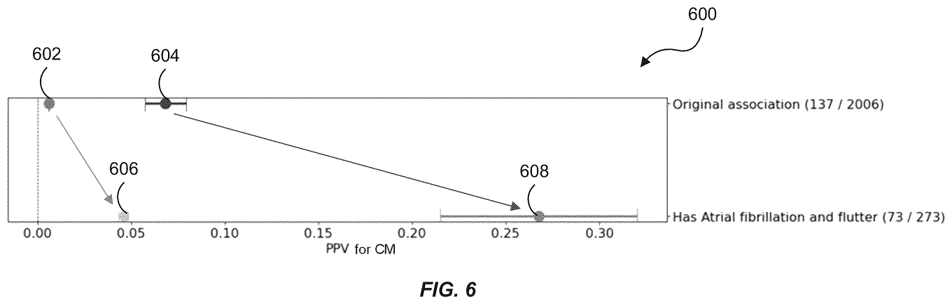

The processing system 106 provides an improved PPV calculation, an example of which is shown in graph 600 of FIG. 6. For example, probands that were not carriers of TTNtvs are illustrated with the reference number 602. Probands that were carriers of TTNtvs are illustrated with the reference number 604. Before the processing system 106 analyzed the interacting phenotypes, the probands of the reference number 602 had a PPV of roughly 0.01, indicating that those probands were not likely to contract cardiomyopathy (e.g., a roughly 1% chance). And the probands of the reference number 604 had a PPV of roughly 0.07, indicating that those programs were more likely to contract cardiomyopathy than the probands of reference number 602, but still not very likely to contract cardiomyopathy (e.g., a roughly 7% chance). However, when the processing system 106 analyzed each of these groups of probands with atrial fibrillation as an interacting phenotype, the PPV increased dramatically. For example, the probands with atrial fibrillation and no TTNtvs had an increase in PPV of almost 4% (reference number 606). And the probands with atrial fibrillation and TTNtvs had an increase in PPV of almost 27% (reference number 608). Thus, when a patient has been diagnosed with atrial fibrillation, a medical professional can better inform the patient of the risks of contracting cardiomyopathy, such that the patient can make more informed choices as to the care the patient would like to receive.

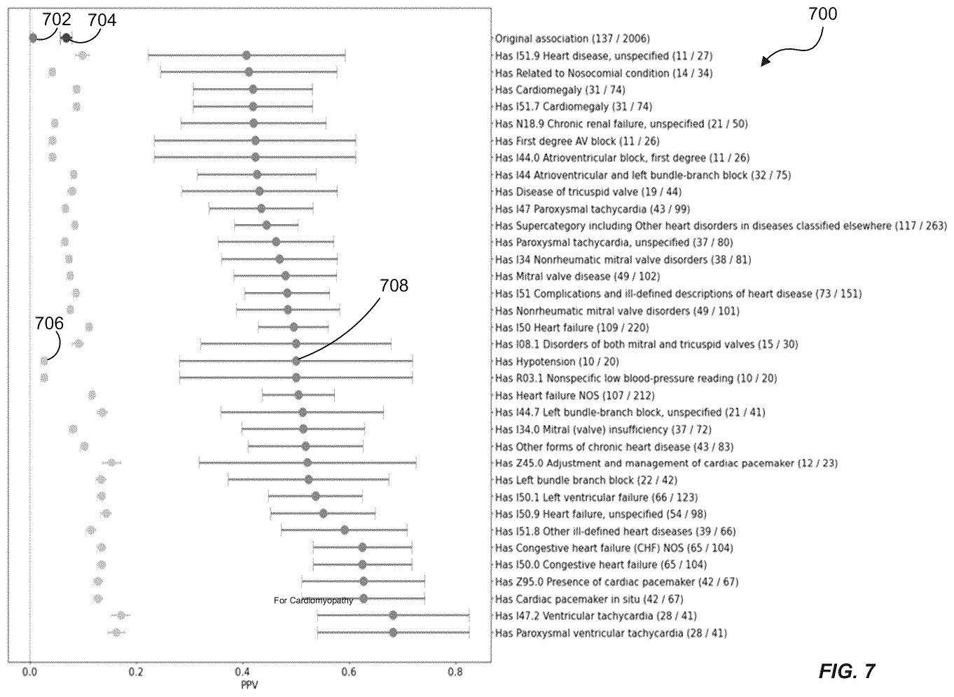

Other examples of PPV improvements are exemplarily shown in the graph 700 of FIG. 7. In this example, probands of reference number 702 again are not carriers of TTNtvs and have a prevalence of roughly 1%, indicating that those probands were not likely to contract cardiomyopathy. And the probands of the reference number 704 are carriers of TTNtvs and have a PPV of roughly 7%, indicating an increased, albeit relatively low, risk of contracting cardiomyopathy. However, when the processing system 106 analyzes each of these groups of probands with various interacting phenotypes, the PPV for each of these groups can increase dramatically. For example, for the probands without TTNtvs 706 but with hypotension as an interacting phenotype, the PPV increased from roughly 1% to 3%. But the probands with TTNtvs 708 and also hypotension increase the PPV to almost 50%, indicating that subsequent patients that have hypotension and are carriers of the TTNtvs have an almost 50% chance of contracting cardiomyopathy.

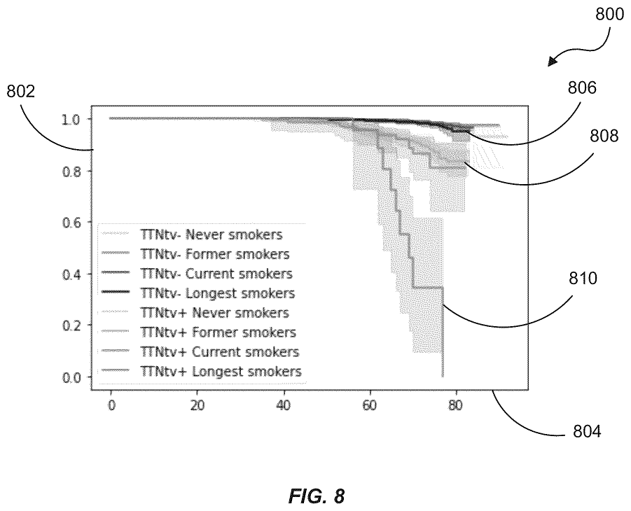

In some embodiments, interacting phenotypes are combined with genetic results and age or other covariates in order to report risk on a granular level. And the risk profile can be dramatic. For example, FIG. 8 is a graph 800 that exemplarily illustrates the risk profiles of carriers and noncarriers of TTNtvs contracting cardiomyopathy. The graph 800 illustrates the proportion of probands without cardiomyopathy (axis 802) versus the age of the probands (axis 804) and the effects of smoking tobacco as an interacting phenotype. In this example, probands associated with the reference number 806 were negative for being carriers of TTNtvs, albeit long-term smokers. Yet probably less than 5% of those probands contracted cardiomyopathy by the age of 80. The probands associated with the reference number 808 were positive for being carriers of TTNtvs and former smokers. Almost 20% of these probands contracted cardiomyopathy by the age of 80. And the probands associated with reference number 810 were positive for being carriers of TTNtvs and the longest smokers. Almost 100% of these probands contracted cardiomyopathy before the age of 80 and almost 50% of these probands contracted cardiomyopathy by the age of 70.

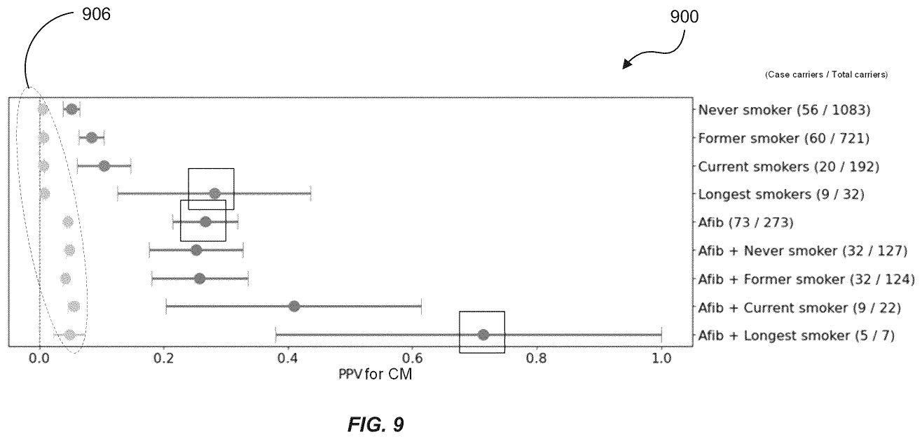

FIG. 9 is a risk profile chart 900 that illustrates the effects of atrial fibrillation and various tobacco smoking habits (i.e., both interacting phenotypes) for probands that were positive for being carriers of TTNtvs and for probands that were not positive for being carriers of TTNtvs. For example, the probands associated with the reference number 906 are not carriers of TTNtvs. As such, smoking has little effect on the PPV for contracting cardiomyopathy in those probands. However, when combined with atrial fibrillation, the PPV for contracting cardiomyopathy increases almost 5% in some instances.

The remaining instances of the probands are carriers of TTNtvs. Again, the probands that are carriers of TTNtvs and have never smoked tobacco have a subtle yet increased PPV of contracting cardiomyopathy over the probands that are not carriers of TTNtvs. However, when smoking tobacco and atrial fibrillation are introduced as interacting phenotypes, the PPV of contracting cardiomyopathy increases significantly. For example, a carrier of TTNtvs who has atrial fibrillation and is a long-term smoker has an almost 75% PPV for contracting cardiomyopathy. Accordingly, when a subsequent patient has tested positive for TTNtvs in a genetic screening and has an interacting phenotype of this risk profile, a medical professional can better recommend a treatment for the patient and the patient can have a more informed opinion regarding that treatment.

The embodiments herein provide notable benefits over previous genetic screening and disease risk associations by detecting combinations of phenotypes that increase the risk of patients that are carriers of a genetic variant. Furthermore, the embodiments herein are processing-efficient in that they are capable of considering the impact of thousands of phenotypes on risk at once, as opposed to considering a few phenotypes at a time (e.g., as required by a formal interaction analyses).

Additionally, unlike formal interaction analyses that are only capable of detecting traits that have a multiplicative impact on risk, the processes herein are also capable of detecting traits that have an additive impact on risk. For example, being a TTNtv carrier and having atrial fibrillation are risk factors for cardiomyopathy. The combination of carrying a TTNtv and having atrial fibrillation does not produce a multiplicatively higher risk for cardiomyopathy, but rather an additive risk. However, this additive combination pushes the PPV for cardiomyopathy up from <10% for either risk factor separately to 35% when combined, putting it into the range of a meaningful risk result.

### EXAMPLES

Laboratory procedures related to genetics may include accessioning, sample plating, storage, extraction, library preparation, enrichment, and sequencing processes. These processes acquire genetic material from a sample, separate the genetic material from other constituents, duplicate the genetic material, and quantify the genetic material order to determine a swathe of sequence data, such as an exome or entire genome for a subject (e.g., a human, an animal, a pathogen, an organelle, etc.).

### Accessioning

Accessioning refers to receiving and preparing samples for later laboratory processes. In one embodiment, accessioning includes receiving a batch of samples (e.g., hundreds or thousands of samples) from a package delivery service each day for processing. For example, packages that each include tens or hundreds of samples may be delivered via the United States Postal Service (USPS), or a private package carrier.

Each sample may be retained within a sample container, such as a five milliliter (mL) test tube. The sample container is sealed to prevent the sample from being exposed to the environment and also to prevent the sample from co-mingling with other samples. For example, the sample may be sealed via a cap that is threaded, glued, press-fit, etc. At the time of delivery, the sample container may further include a remnant of a sampling tool, such as a portion of a swab that was utilized to acquire the sample.

In many embodiments, a Customer Sample Identifier (CSI) is reported via a component affixed to or integrated with the sample container. The CSI uniquely distinguishes the sample from other samples being received. For example, a CSI may uniquely distinguish a sample from other samples in the same batch, other samples received on the same date, other samples received from the same customer, etc. The CSI may be reported via a barcode label, Quick Response (QR) code label, Radio Frequency Identifier (RFID) chip, or any suitable visual, transmission-generating, or other physical component affixed to or integrated with the sample container.

In further embodiments, the sample container is itself sealed within an external container such as a bag. Using an external container helps to prevent contamination, by ensuring that a technician at the laboratory does not contact biological material from the sample that may exist on an outer surface of the sample container. Use of an external container may be required by law (e.g., Department of Transportation (DOT) guidelines). Use of an external container additionally helps to prevent cross-contamination between samples. Furthermore, in embodiments where samples may include blood or a pathogen, an external container provides an additional barrier to protect the health of technicians. The external container may additionally include documentation confirming the CSI, information for the subject that the sample was sourced from, and/or information indicating circumstances of sampling. The circumstances of sampling may include, for example, a sampling date, a sampling method, a location that the sample was acquired, a name or title for a person who performed the sampling, and/or additional notes.

In this embodiment, the sample comprises a chemical solution. For example, the sample may comprise a prepared aqueous solution such as a saline solution, or may comprise a bodily fluid such as blood, saliva, mucus, etc. In some embodiments each of the samples fills between two and five milliliters of volume within the sample container.

The sample further includes genetic material such as Deoxyribonucleic Acid (DNA), Ribonucleic Acid (RNA), etc. In many instances, the genetic material is one of many constituent components within the sample. For example, the genetic material may exist within the nuclei of white blood cells that are included within the sample. In a further example, genetic material may exist within viruses or bacteria within the sample. In this embodiment, the genetic material is not yet isolated from the remaining constituent components of the sample.

After receipt of the samples, batches of the samples (e.g., as stored within sample containers and/or external containers) may be heated in ovens to facilitate cell lysis. The temperature, and duration of heating, may be chosen such that pathogenic material within the samples is rendered harmless, or such that cellular lysis occurs. For example, heating may occur at a temperature of between forty and eighty (e.g., fifty) degrees Celsius (C), for a period of time between fifteen and two hundred (e.g., thirty) minutes. In some embodiments, including embodiments wherein the samples are the contents of a blood draw, the heating step may be foregone.

Upon completion of heating, the batches of samples are removed from the ovens. In one embodiment, sample containers are removed from corresponding external containers, such as by cutting the external containers open. With the sample containers now available for direct interaction, the sample containers are inspected. As a part of this process, a technician or automated system may determine the CSI for the sample, and may compare the CSI to a CSI listed on the documentation provided in the external container. If there is a discrepancy between the CSIs on the sample container and in the documentation, the sample may be flagged as having an error condition. Similarly, if the CSI on the sample container is damaged (e.g., abraded, heat-damaged, or water-damaged) and has become unreadable, the sample may be flagged as having an error condition.

The technician or automated system further inspects the contents of the sample container, via visual or other methods. If the sample does not include expected constituent components, then the sample is flagged as having an error condition. For example, if the sample includes a fluid that is not permitted (e.g., blood), includes an entire swab or no swab, appears to have a fractured or broken casing, or is outside of an expected range of volume (e.g., between two and five milliliters), then the sample may be flagged as having an error condition.

Samples that have not been flagged as having an error condition proceed to sample integration. As a part of sample integration, the sample is assigned a Laboratory Sample Identifier (LSI). The LSI uniquely identifies the sample from other samples received for the batch, received on the same day, processed in the same laboratory, and/or handled by the same company performing sequencing. In many embodiments, the LSI is stored in a laboratory sample database, and is uniquely associated with a corresponding CSI for the sample. The LSI is also associated with any error conditions reported for the sample.

In many embodiments, CSIs originally provided with the samples are in the form of a paper barcode. In such embodiments, the paper barcode may be printed in aqueous ink. This renders the barcode subject to degradation upon exposure to liquid in the laboratory environment, which is undesirable.

To ensure that each sample container is capable of traveling through the laboratory without its identifier being physically degraded, a corresponding LSI may be indicated at the sample container. The LSI may be indicated via a barcode label, Quick Response (QR) code, Radio Frequency Identifier (RFID) chip, or other visual, transmission-generating, or other physical component affixed to or integrated with the sample container.

In one embodiment, the LSI is printed onto a barcode label comprising rip-proof material (e.g., vinyl) in a water insoluble ink. This implementation ensures that the barcode label is resistant to physical and chemical degradation. The barcode may be applied around an entire perimeter of the sample container, ensuring that the sample container may be scanned from any angle.

In further embodiments, the element used to report the LSI is accompanied by a visually distinct mark that enables rapid confirmation by a technician that the sample has been integrated into the laboratory environment. The visually distinct mark may comprise a colored ring (e.g., around an entire perimeter of the sample container), a logo, a physical feature, a stamp, etc.

### Sample Plating

With the samples having been successfully integrated into the laboratory environment, the samples are ready for analytics to be performed. To this end, the samples are prepared for transfer to a sample microplate. The sample microplate may be labeled with a unique identifier similar to the techniques used for sample containers above. The unique identifier distinguishes the sample microplate from other sample microplates. In one embodiment, the sample microplate comprises a solid body defining three hundred and eighty four wells, distributed across sixteen rows and twenty-four columns, each well having a capacity of between thirty and one hundred microliters. In a further embodiment, the sample microplate comprises a solid body defining ninety six wells, distributed across eight rows and twelve columns, each well having a capacity of between one hundred and three hundred microliters.

As a part of preparing the samples for transfer to the sample microplate, a technician may place sample containers onto a rack, and scan each sample container to determine an LSI for each location on the rack. In some embodiments, the rack is assigned a unique identifier that distinguishes it from other racks. The rack may be labeled with a unique identifier similar to the techniques used for sample containers. The technician associates the unique identifier for the rack, along with the locations assigned to the samples, with the corresponding LSIs of the samples stored at the rack.

The technician additionally unseals the sample containers. Unscaling of sample containers may be a deeply labor-intensive process, particularly when laboratory processes are performed at scale to handle tens of thousands of samples per day. Thus, a technician may utilize automated tooling to enhance the speed at which sample containers are unsealed. The tooling may, for example, unscrew, cut, or drill each sample container, in order to make the sample within available for physical transfer to the sample microplate.

One or more racks of samples are provided to a Liquid Handler (LH), such as an automated robot that operates an end effector in accordance with one or more Numerical Control (NC) programs to transfer liquids between wells via arrays of micropipettes. An LH is also known as a “Liquid Handling System.” One example of an LH is the Hamilton Microlab Star Liquid Handling System.

In this embodiment, the LH proceeds to transfer a portion of each sample at the rack to a unique predetermined well within the sample microplate. The LH transfers the portions of the samples to the wells of the sample microplate by providing instructions to actuators, piezoelectric elements, and/or pressure systems operating the end effector. The end effector aligns its array of micropipettes with the sample containers to retrieve portions of the samples. The end effector then dynamically aligns its array of micropipettes with the sample microplate to deposit the portions of the samples at the predetermined wells.

Because there is a known relationship between rack locations and wells of the sample microplate (e.g., as indicated by row and column), the laboratory sample database may be updated to indicate the well storing genetic material for each sample. The laboratory sample database may further be updated to associate a unique identifier for the sample microplate with the samples stored therein.

In one embodiment, programmed instructions for the LH may direct the end effector to position itself above a set of disposable tips, descend into the tips to attach the tips, reposition the end effector above the rack of sample containers, adjust spacing between micropipettes within the array, descend until the tips reach the sample containers, draw liquid from the sample containers, deposit the liquid into a well at the well plate, and then dispose of the tips. Such a process may be repeated across sample containers stored on multiple racks until the sample microplate is filled with portions from the samples. In one embodiment, one or more wells on the sample microplate are filled with a control reagent instead of a portion of a sample.

The amount of liquid drawn from each sample container may comprise a small fraction of the overall volume of the sample container. For example, an amount of liquid drawn may comprise several microliters, such as between two and ten microliters. Upon completion of transfer from the sample containers to the wells, the sample microplate may be covered with a liquid and/or gas-impermeable layer, such as foil or paraffin. Sample containers remaining on the racks may be rescaled, for example with pressure-fit caps having a color distinct from an original color for the sample containers. With accessioning now complete for the sample microplate, the sample microplate is transferred to a next section of the laboratory for processing.

### Storage

In one embodiment, accessioned samples, samples ready for analytics, and/or samples that have already been sequenced, are stored for later use. For example, samples, sample containers, and/or microplates may be stored at room temperature, or may be cryogenically frozen at a low temperature (e.g., negative eighty degrees Celsius) and arranged in racks for later retrieval. Samples may be preserved for periods of days or years, enabling rapid re-testing to be performed for subjects without the need for re-acquiring genetic material. The stored samples provide notable value in the event that contents of a well or microplate do not meet with rigorous quality control standards.

### Extraction

Sample microplates are transferred to a portion of the laboratory dedicated to extraction of the genetic material. The segment of the laboratory that performs extraction and other pre-amplification operations may be sealed from, and/or positively pressurized relative to, other portions of the laboratory.

During extraction, a sample microplate is acquired and provided to an LH. The LH may apply a reagent to each well that lyses cells within each well. For example, this may be performed in order to lyse white blood cells containing genetic material for a human, or may comprise lysing other types of cells to expose other types of genetic material. The reagents used for pre-amplification processes may be stored at the LH in a temperature controlled manner, and may even be vibrated or mixed on a regular basis to ensure that the reagents are evenly distributed in suspension.

Extraction further includes an LH aspirating and dispensing reagents that selectively bind to genetic material released from the lysed cells. This process may include applying a bead to the well. In one embodiment, the beads comprise magnetic beads that selectively bind to the genetic material (e.g., DNA). This allows for isolation and purification of the genetic material while contaminants remain in solution. In one embodiment, the magnetic bead is drawn to a magnetic base at or under the sample microplate. After the genetic material has been drawn to the bead, and after the bead has been secured to the base of the well, a flushing step may be performed wherein remaining fluid in each well is washed away. This ensures that potential impurities are removed from the well. The LH may further add or remove fluid from each well to perform additional concentration and/or elution of the genetic material, and may transfer fluid from the wells of the sample microplate to a genome stock microplate. The genome stock microplate may be labeled with a unique identifier, and the contents of each well of the genome stock microplate may be associated with a corresponding LSI. In all phases of operation, the LH is operated to ensure that fluid is not transferred between wells, as this results in contamination.

In one embodiment, a portion of fluid is removed from each well of the genome stock microplate for quality control purposes. Concentration of genetic material within the wells may be confirmed via testing of this fluid, such as by application of a dye that reacts with the genetic material at known levels of fluorescence for known concentrations.

### Library Preparation

After extraction is completed, library preparation is performed for the contents of the genome stock microplate. The bead for each well, including ionically bonded genetic material, is transferred to a distinct well of a library preparation microplate. The library preparation microplate includes an identifier that uniquely distinguishes it from other library preparation microplates, and the LSI associated with each well on the sample microplate may be mapped to a corresponding well on the library preparation microplate.

The library preparation microplate may be transferred to a new portion of the laboratory that is sealed from, and/or positively pressurized relative to, other portions of the laboratory that do not perform amplification of genetic material. This feature helps to prevent amplified genetic material from entering portions of the laboratory where genetic material has not been amplified, which could result in contamination. The transfer process may be performed by placing the well plate into an airlock at the pre-amplification portion of the laboratory, sealing the airlock, and then retrieving the well plate from the airlock via the amplification portion of the laboratory.

A reagent is applied to each well of the library preparation microplate. The reagent ionically bonds to the surface of the bead within the well, and does so more strongly than the genetic material. This releases the genetic material from the surface of the bead of each well, enabling the genetic material to be chemically interacted with.

Library preparation may include normalization of a concentration of genetic material in each well of the sample microplate. Library preparation further includes fragmentation of the genetic material via an enzyme or via the application of physical forces. During this process, the entire genome (e.g., roughly three billion base pairs for a human genome), may be fragmented into pieces. In one embodiment, the pieces vary between three hundred and four hundred base pairs in length. These pieces are known as nucleic acid fragments.

The nucleic acid fragments undergo adaptor ligation and indexing in accordance with known techniques. For example, this may comprise Next Generation Sequencing (NGS) library preparation processes defined by Illumina. Next, a limited amount of Polymerase Chain Reaction (PCR) amplification is performed upon the library. The resulting solution is then purified and eluted via operation of the LH.

During library preparation, one or more reference samples of genetic material, distinct from the genetic material found in the samples, may be added to wells of the library preparation microplate. The reference samples do not include genetic material received from a customer, but rather include known sequences of base pairs. The reference samples serve as controls to ensure that processes are carried out with sufficient quality.

Upon completion of library preparation, desired fragments of the genetic material (e.g., thousands or millions of distinct fragments of the genetic material, each corresponding with a different portion of a genome of the subject) have been ligated to predefined adapters (e.g., DNA adapters) that bind with the genetic material. Each of the adaptor ligated fragments is referred to as a “library.”

In further embodiments, the probes applied to each well of the well plate include chemical identifiers (colloquially referred to as “barcodes”) that are distinct from each other. The use of a different chemical identifier for probes applied to each well of the well plate enables sequencing to later be performed for multiple subjects on the same flow cell, without conflating sequencing results for those subjects.

The library preparation process may further comprise controlling a concentration of the genetic material in each well, and purification and/or elution of the resulting material. Similar to the processes performed after extraction of genetic material, concentration of genetic material after library preparation may be confirmed for each well via testing.

### Enrichment

After library preparation, enrichment processes may be performed in order to either directly amplify (e.g., via amplicon or multiplexed PCR) or capture (e.g., via hybrid capture) predefined libraries. This enhances the case of sequencing desired portions of the genome.

During enrichment, customized biotinylated oligonucleotide probes are applied to the libraries. The probes selectively hybridize genetic material occupying desired portions of the genome for the genetic material, such as specific genes, or the entire exome. Magnetic beads bind to biotin molecules in the probes to attach the hybridized material to the magnetic beads. Magnetic forces capture the beads in place, enabling remaining fluid within each well to be removed or washed out, thereby removing impurities and leaving only the genetic material that is desired. Genetic material may be released from the beads in a similar manner to that discussed above for prior processes.

In one embodiment, hybrid capture target enrichment is performed. During this process, the probes comprise tailored oligonucleotides that are chosen to bind to the genetic material. The range of probes may be tailored as a group to bind to specific alleles, specific genes, the exome, the entire genome, etc. That is, each probe may bind to a nucleic acid fragment at a specific location on the genome, and the range of probes may be selected to ensure that alleles, genes, the exome, or the entire genome of the subject being considered is acquired. Utilizing probes in this manner may enhance efficiency of the sequencing process, by foregoing the need to sequence all of the roughly three billion base pairs found in the human genome.

The enrichment process may further comprise controlling a concentration of the genetic material in each well, and purification and/or elution of the resulting material. Similar to the processes performed after extraction of genetic material, concentration of genetic material after enrichment may be confirmed for each well via testing.

### Sequencing

Sequencing may be performed according to any of a variety of techniques, including short-read and long-read techniques. In one embodiment, the sequencing is performed as Sequencing by Synthesis (SBS) at genetic analyzer equipment. For example, sets of enriched libraries of genetic material bound to probes in earlier steps may be transferred to a flow cell, and annealed to oligonucleotide probes within the flow cell. At this stage, the contents of multiple wells may be applied to the same flow cell, because the libraries within those wells are tagged with the chemical identifiers referred to above. In one embodiment, the chemical identifiers comprise nucleotide sequences that are detectable during the sequencing process to determine a corresponding LSI.

Complementary sequences may then be created via enzymatic extension to create a double-stranded portion of genetic material. The double-stranded genetic material may then be denatured, and the library fragment may be washed away. Bridge amplification may then be performed to create copies of the remaining molecule in a localized cluster. For example, a cluster may comprise twenty to fifty copies of the same molecule, localized to a location the size smaller than a pinhead on the flowcell.

Sequencing primers are annealed to library adapters in order to prepare the flow cell for SBS. During SBS, the sequencing primer uses reverse terminator fluorescent oligonucleotides, one base per cycle, for a number of cycles (e.g., one hundred and fifty cycles) in the forward direction. After the addition of each nucleotide, clusters are excited by a light source, resulting in fluorescence which can be measured. The emission wavelength and signal intensity for each cluster determines a base call for that cluster. Fluorescent moieties are then flushed from the flow cell. A chemical group blocking a 3′ end of the fragment is then removed, enabling a subsequent nucleotide to be read. This tightly controls nucleotide addition and detection.

Base calls across cycles at the same physical location on the flow cell occur at the same cluster, and hence indicate sequential reads for copies of the same fragment of the genetic material. After each cycle, denaturing and annealing are performed to extend the index primer. A complementary reverse strand is created and extended via bridge amplification. The reverse strand is then read in the reverse direction for a number of cycles, in a manner similar to reads in the forward direction.

Depending on whether a complete human genome, or another set of genomic data, is being tested, different reagents (e.g., probes, primers, etc.) may be chosen. That is, different reagents may be utilized for library preparation for a pathogen (e.g., bacteria, virus) or an organelle (e.g., mitochondria) than for a human genome. Pathogens exhibiting Ribonucleic Acid (RNA) genomes may have their genetic material translated to DNA before sequencing, enrichment, and/or library preparation are performed, via known techniques, such as Next Generation Sequencing (NGS) techniques.

Throughout the processes discussed above, the laboratory environment may be carefully controlled to ensure quality. For example, temperature within each segment of the laboratory may be carefully monitored and controlled, and ultraviolet lighting or other features capable of inactivating genetic material may be carefully positioned to ensure that contamination does not occur.

In some embodiments, genetic material is used for detection of a pathogen rather than for sequencing. Detecting a pathogen may involve the use of a real-time PCR system that performs PCR. The real-time PCR system may further add a reactive agent to individual wells of a library preparation microplate, that fluoresces when bound to genetic material for the pathogen. By analyzing fluorescence at known periods of time after PCR has initiated, presence of a pathogen is determined. Genetic testing for a pathogen may thereby forego sequencing in some embodiments.

### Bioinformatics

Raw sequencing data generated during synthesis is stored in a file format such as Binary Base Call (BCL). This raw data may be fed to an analytical pipeline such as a cloud-based computing environment. Raw sequencing data may be processed by the pipeline into a second format, such as a text-based FASTQ format, that reports quality scores. The second format is then analyzed to perform alignment of sequence reads to a reference genome, such as a reference genome reported in a Browser Extensible Data (BED) file. The aligned sequence data may be reported as a Binary Alignment Map (BAM) file. The aligned sequence data may then be called, resulting in a Variant Call Format (VCF) file reporting called variants at each location of the genome that was sequenced, together with secondary metrics such as quality indicator metrics.

The called sequence data may be provided to a data analyst via a User Interface (UI), such as a Graphical User Interface (GUI) presented via a display. The technician may then validate the resulting called sequence data and release it for reporting to subjects, health care providers, and/or scientists.

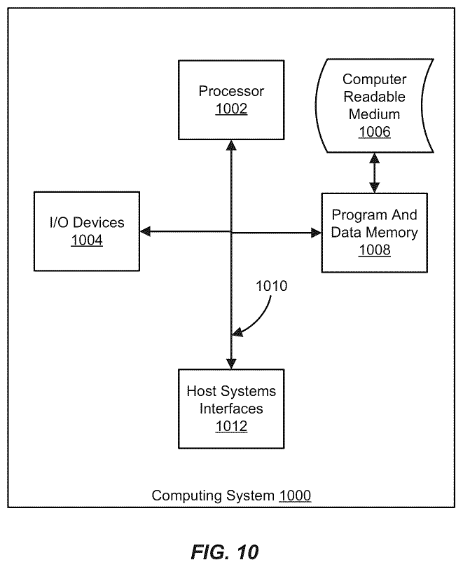

Any of the above embodiments herein may be rearranged and/or combined with other embodiments. Accordingly, the concepts herein are not to be limited to any particular embodiment disclosed herein. Additionally, the embodiments can take the form of entirely hardware or comprising both hardware and software elements. Portions of the embodiments may be implemented in software, which includes but is not limited to firmware, resident software, microcode, etc. FIG. 10 illustrates a computing system 1000 in which a computer readable medium 1006 may provide instructions for performing any of the methods disclosed herein.

Furthermore, the embodiments can take the form of a computer program product accessible from the computer readable medium 1006 providing program code for use by or in connection with a computer or any instruction execution system. For the purposes of this description, the computer readable medium 1006 can be any apparatus that can tangibly store the program for use by or in connection with the instruction execution system, apparatus, or device, including the computing system 1000.

The computer readable medium 1006 can be any tangible electronic, magnetic, optical, electromagnetic, infrared, or semiconductor system (or apparatus or device). Examples of a computer readable medium 1006 include a semiconductor or solid state memory, magnetic tape, a removable computer diskette, a random access memory (RAM), NAND flash memory, a read-only memory (ROM), a rigid magnetic disk and an optical disk. Some examples of optical disks include compact disk-read only memory (CD-ROM), compact disk-read/write (CD-R/W) and digital versatile disc (DVD).

The computing system 1000, suitable for storing and/or executing program code, can include one or more processors 1002 coupled directly or indirectly to memory 1008 through a system bus 1010. The memory 1008 can include local memory employed during actual execution of the program code, bulk storage, and cache memories which provide temporary storage of at least some program code in order to reduce the number of times code is retrieved from bulk storage during execution. Input/output or I/O devices 1004 (including but not limited to keyboards, displays, pointing devices, etc.) can be coupled to the system either directly or through intervening I/O controllers. Network adapters may also be coupled to the system to enable the computing system 1000 to become coupled to other data processing systems, such as through host systems interfaces 1012, or remote printers or storage devices through intervening private or public networks. Modems, cable modem and Ethernet cards are just a few of the currently available types of network adapters.

What is claimed is:

1. A method, comprising:
identifying, from a larger population of gene sequences, a first plurality of gene sequences of probands having a genetic variant that is susceptible to contracting a primary phenotype;
for each of a plurality of interacting phenotypes:
identifying, from the first plurality of gene sequences, a second plurality of gene sequences of probands having the interacting phenotype;
determining a difference between an odds ratio indicating a likelihood of probands having the primary phenotype for the second plurality of gene sequences and an odds ratio indicating a likelihood of probands having the primary phenotype for the first plurality of gene sequences; and
selecting the interacting phenotype based on the difference; and
identifying a risk of contracting the primary phenotype for each of the selected interacting phenotypes.

2. The method of claim 1, further comprising:
processing a gene sequence of a patient to determine that the gene sequence of the patient has the genetic variant that is susceptible to contracting the primary phenotype; and
determining a risk of the patient contracting the primary phenotype based on whether the patient has a selected interacting phenotype.

3. The method of claim 1, further comprising:
subdividing an interacting phenotype by:
determining a mean of a probability distribution of the interacting phenotype for the first plurality of gene sequences;
defining a first interacting phenotype comprising values outside one standard deviation higher than the mean; and
defining a second interacting phenotype comprising values outside one standard deviation lower than the mean.

4. The method of claim 1, further comprising:
selecting the interacting phenotype if a positive predictive value of the interacting phenotype contributing to the primary phenotype breaches a pre-determined threshold; and
selecting the interacting phenotype if a negative predictive value of the interacting phenotype contributing to the primary phenotype breaches another pre-determined threshold.

5. The method of claim 1, further comprising:
including an additional interacting phenotype to identify the second plurality of gene sequences for selection.

6. The method of claim 5, further comprising:
computing an odds ratio for the additional interacting phenotype.

7. The method of claim 1, further comprising:
performing a regression analysis to compute the odds ratios.

8. A non-transitory computer readable medium embodying programmed instructions which, when executed by a processor, are operable for performing a method comprising:
identifying, from a larger population of gene sequences, a first plurality of gene sequences of probands having a genetic variant that is susceptible to contracting a primary phenotype;
for each of a plurality of interacting phenotypes:
identifying, from the first plurality of gene sequences, a second plurality of gene sequences of probands having the interacting phenotype;
determining a difference between an odds ratio indicating a likelihood of probands having the primary phenotype for the second plurality of gene sequences and an odds ratio indicating a likelihood of probands having the primary phenotype for the first plurality of gene sequences; and
selecting the interacting phenotype based on the difference; and
identifying a risk of contracting the primary phenotype for each of the selected interacting phenotypes.

9. The computer readable medium of claim 8, wherein the method further comprises:
processing a gene sequence of a patient to determine that the gene sequence of the patient has the genetic variant that is susceptible to contracting the primary phenotype; and
determining a risk of the patient contracting the primary phenotype based on whether the patient has a selected interacting phenotype.

10. The computer readable medium of claim 8, wherein the method further comprises:
subdividing an interacting phenotype by:
determining a mean of a probability distribution of the interacting phenotype for the first plurality of gene sequences;
defining a first interacting phenotype comprising values outside one standard deviation higher than the mean; and
defining a second interacting phenotype comprising values outside one standard deviation lower than the mean.

11. The computer readable medium of claim 8, wherein the method further comprises:
selecting the interacting phenotype if a positive predictive value of the interacting phenotype contributing to the primary phenotype breaches a pre-determined threshold; and
selecting the interacting phenotype if a negative predictive value of the interacting phenotype contributing to the primary phenotype breaches another pre-determined threshold.

12. The computer readable medium of claim 8, wherein the method further comprises:
including an additional interacting phenotype to identify the second plurality of gene sequences for selection.

13. The computer readable medium of claim 12, wherein the method further comprises:
computing an odds ratio for the additional interacting phenotype.

14. The computer readable medium of claim 8, wherein the method further comprises:
performing a regression analysis to compute the odds ratios.

15. A system, comprising:
a database of gene sequences of probands; and
a processor that:
identifies, from a larger population of the gene sequences, a first plurality of gene sequences of probands having a genetic variant that is susceptible to contracting a primary phenotype;
for each of a plurality of interacting phenotypes:
identifies, from the first plurality of gene sequences, a second plurality of gene sequences of probands having the interacting phenotype;
determines a difference between an odds ratio indicating a likelihood of probands having the primary phenotype for the second plurality of gene sequences and an odds ratio indicating a likelihood of probands having the primary phenotype for the first plurality of gene sequences; and
selects the interacting phenotype based on the difference; and
identifies a risk of contracting the primary phenotype for each of the selected interacting phenotypes.

16. The system of claim 15, wherein the processor further:
processes a gene sequence of a patient to determine that the gene sequence of the patient has the genetic variant that is susceptible to contracting the primary phenotype; and
determines a risk of the patient contracting the primary phenotype based on whether the patient has a selected interacting phenotype.

17. The system of claim 15, wherein the processor further:
subdivides an interacting phenotype by:
determines a mean of a probability distribution of the interacting phenotype for the first plurality of gene sequences;
defines a first interacting phenotype comprising values outside one standard deviation higher than the mean; and
defines a second interacting phenotype comprising values outside one standard deviation lower than the mean.

18. The system of claim 15, wherein the processor further:
selects the interacting phenotype if a positive predictive value of the interacting phenotype contributing to the primary phenotype breaches a pre-determined threshold; and
selects the interacting phenotype if a negative predictive value of the interacting phenotype contributing to the primary phenotype breaches another pre-determined threshold.

19. The system of claim 15, wherein the processor further:
includes an additional interacting phenotype to identify the second plurality of gene sequences for selection.

20. The system of claim 19, wherein the processor further:
computes an odds ratio for the additional interacting phenotype.

21. The system of claim 15, wherein the processor further:
performs a regression analysis to compute the odds ratios.
